(19) United States
(12) Patent Application Publication
(10) Pub. No.: US 2025/0050000 A1
(43) Pub. Date: Feb. 13, 2025

(54) WEIGHT-BASED SYSTEMS FOR USE IN EXTRACORPOREAL BLOOD TREATMENT

(71) Applicants: BAXTER INTERNATIONAL INC., Deerfield, IL (US); BAXTER HEALTHCARE SA, Glattpark (Opfikon) (CH)

(72) Inventors: Olof JANSSON, Vellinge (SE); Michael PETTERSSON, Malmö (SE); Thomas HERTZ, Lund (SE); Jonas FORS, Malmö (SE); Per-Olof BORGQVIST, Lund (SE)

(21) Appl. No.: 18/719,082

(22) PCT Filed: Dec. 14, 2022
(86) PCT No.: PCT/EP2022/085786

§ 371 Date: Jun. 12, 2024

(30) Foreign Application Priority Data
Dec. 15, 2021 (SE) 2130364-9

(51) Int. Cl. A61M 1/16

(52) U.S. Cl. CPC A61M 1/1643; A61M 1/1603

(57) ABSTRACT

Ultrafiltration (UF) is monitored in a weight-based system for extracorporeal blood treatment. The system comprises a container holding a fluid that is pumped to or from a dialyzer by a pump. The container is intermittently refilled or drained by an adjustment arrangement during a level adjustment period (LAP). A scale measures the weight of the container. A monitoring device is operated to determine, before and after the LAP while the adjustment arrangement is deactivated and the pump is operated at a known setting, first and second values of a parameter of the pump based on a weight signal from the scale. The parameter may be flow rate or stroke volume. The monitoring device estimates a time profile for the parameter during the LAP based on the first and second values and determines the UF parameter based on the time profile.

### TECHNICAL FIELD

The present disclosure relates to systems in the field of extracorporeal blood treatment and in particular to such systems comprising one or more weight scales for monitoring fluid flow into and out of a container.

### BACKGROUND ART

In treating renal failure, various methods of purification and treatment of blood with machinery are used to replace the function of a healthy kidney. Such methods include extracorporeal (EC) blood treatment and aim at withdrawing fluid and removing substances from the blood, and they may also involve adding fluid and substances to the blood. In EC blood treatment, a dialysis fluid is pumped through a blood filtration unit, commonly denoted dialyzer, in which fluid and substances are transported over a semi-permeable membrane. Common modalities of EC blood treatment include hemodialysis, hemofiltration and hemodiafiltration.

Machines for EC blood treatment are often separated into machines for treatment of patients with chronic kidney disease (CKD), commonly known as “chronic dialysis”, and machines for treatment of patients with acute kidney injury (AKI), commonly known as “acute dialysis”. Acute dialysis is typically performed continuously or semi-continuously. Continuous therapy is a 24-hour treatment, whereas semi-continuous therapy may be performed daily with a duration of 6-12 hours or more.

Machines for acute treatment commonly comprise scales, on which containers or “fluid bags” are releasably arranged. The operation of the machine is controlled based on the weight of the fluid bags, given by the readings of the scales. Commonly, at least one fluid bag is arranged to hold a fresh dialysis fluid (“dialysis fluid bag”), which is used in the dialysis treatment, and at least one fluid bag is arranged to receive spent dialysis fluid (“effluent bag”). As is well-known to the skilled person, EC blood treatment involves extracting excess fluid from the patient, commonly known as “ultrafiltration”. The excess fluid is included in the spent dialysis fluid. The ultrafiltration is controlled by a supply pump and an effluent pump in the machine. The amount of excess fluid extracted from the patient and the rate of extraction are important treatment parameters during dialysis. In operation, the machine calculates and monitors these treatment parameters based on the readings of the scales and controls the supply and effluent pumps to achieve corresponding set values.

During EC blood treatment, the dialysis fluid bag will eventually be depleted of fresh dialysis fluid and the effluent bag will be full of spent dialysis fluid. To avoid frequent changes of fluid bags, it has been proposed to configure the machine to intermittently pump fresh dialysis fluid into the dialysis fluid bag from a source, to raise the level of fresh dialysis fluid, and to intermittently pump spent dialysis fluid out of the effluent bag, to lower the level of spent dialysis fluid. Systems with such level adjustment are disclosed in JPH09-239024 and EP3238761. During the level adjustment, ultrafiltration cannot be monitored by use of the scales. One solution to this problem is to stop the flow of dialysis fluid through the dialyzer during the level adjustment. Another solution, proposed in JPH09-239024, is to fix the speeds of the supply and effluent pumps during the level adjustment and quantify the ultrafiltration during the level adjustment under the assumption that the resulting flow rates remain constant throughout the level adjustment.

### SUMMARY

It is an objective to at least partly overcome one or more limitations of the prior art.

A further objective is to improve monitoring of ultrafiltration in a weight-based system for extracorporeal blood treatment.

Another objective is to improve monitoring of ultrafiltration during level adjustment of fluid in a container within such a weight-based system.

Yet another objective is to improve control of ultrafiltration in such a weight-based system.

One or more of these objectives, as well as further objectives that may appear from the description below, are at least partly achieved by devices, a system, computer-implemented methods and a computer-readable medium according to the independent claims, embodiments thereof being defined by the dependent claims.

A first aspect is a device for monitoring ultrafiltration in a system for extracorporeal treatment of blood. The device comprises an input interface, which is configured to receive a measurement signal representative of a momentary weight of a container in the system, the container being connected on a first fluid path to a first dialyzer port for dialysis fluid on a dialyzer for extracorporeal treatment of blood, a pumping device being arranged in the first fluid path, the container being further connected on a second fluid path to an adjustment arrangement for level adjustment of a fluid in the container. The device further comprises processor circuitry, which is connected to the input interface and configured to perform a monitoring procedure in relation to a level adjustment period, in which the adjustment arrangement is selectively activated to perform said level adjustment and the pumping device is operated at a known setting. The processor circuitry, in the monitoring procedure, is configured to: determine, during a first measurement period prior to the level adjustment period and based on the measurement signal, a first value of a pumping parameter of the pumping device while the adjustment arrangement is deactivated and the pumping device is operated at the known setting; determine, during a second measurement period subsequent to the level adjustment period and based on the measurement signal, a second value of the pumping parameter while the adjustment arrangement is deactivated and the pumping device is operated at the known setting; estimate, based on the first and second values, a time profile of the pumping parameter in the level adjustment period; and determine an ultrafiltration parameter for the level adjustment period based on the time profile.

In some embodiments, the processor circuitry is configured to estimate the time profile by performing a linear interpolation of the first and second values.

In some embodiments, the pumping parameter is one of a flow rate generated by the pumping device or a stroke volume of the pumping device.

In some embodiments, the pumping parameter is a stroke volume of the pumping device, and the processor circuitry is further configured to: receive, via the input interface, a further measurement signal representative of a speed of the pumping device, and determine the first and second values of the pumping parameter based on the measurement signal and the further measurement signal during the first and second measurement period, respectively.

In some embodiments, the processor circuitry is further configured to: determine a fluid flow parameter for the level adjustment period, based on the time profile and the further measurement signal during the level adjustment period, and determine the ultrafiltration parameter based on the fluid flow parameter and the fluid flow data.

In some embodiments, the fluid flow parameter represents a total amount of fluid pumped by the pumping device during the level adjustment period.

In some embodiments, the processor circuitry is configured to: determine, based on the further measurement signal, a count of pumping strokes performed by the pumping device during the level adjustment period, calculate an average stroke volume for the level adjustment period based on the time profile, and determine the fluid flow parameter as a function of the average stroke volume and the count of pumping strokes.

In some embodiments, the further measurement signal comprises a predefined number of pulses for each pumping stroke of the pumping device.

In some embodiments, the ultrafiltration parameter comprises an amount of ultrafiltrate extracted from blood in the dialyzer.

In some embodiments, the processor circuitry is configured to: receive, via the input interface, an input signal which is representative of fluid pressure in the first fluid path between the container and the pumping device or which is representative of a control signal for the pumping device, and estimate the time profile based on the first and second values and the input signal during the level adjustment period.

In some embodiments, the processor circuitry is further configured to: obtain calibration data representing a relation between the fluid pressure and the pumping parameter; and estimate the time profile by converting, by use of the calibration data, the input signal received during the level adjustment period into values of the pumping parameter.

In some embodiments, the processor circuitry is further configured to monitor the measurement signal for detection of a step-change during the level adjustment period, and to initiate, upon detection of the step-change, a dedicated action.

In some embodiments, the dedicated action comprises one or more of: a modification of the level adjustment period, accounting for the step-change when estimating the time profile of the pumping parameter, or a modification of the pumping speed of the pumping device.

In some embodiments, the modification of the level adjustment period results in a stop of the level adjustment.

In some embodiments, the processor circuitry is configured to, upon the detection of the step-change, determine a magnitude of the step-change, wherein the dedicated action uses the magnitude.

In some embodiments, the modification of the pumping speed is based on the magnitude to at least partly compensate for a change in flow rate in the first fluid line resulting from the step-change.

In some embodiments, the processor circuitry is configured to estimate the time profile of the pumping parameter based on the first and second values, the magnitude, and a timing of the step-change during the level adjustment period.

In some embodiments, the processor circuitry is configured to: generate, based on the measurement signal, a monitoring signal that represents momentary change of the weight of the container; and detect the step-change in the monitoring signal.

In some embodiments, the pumping device, when operated at the known setting, is configured to repeatedly generate a temporal variation in fluid flow rate in the first fluid line, and the processor circuitry is configured to detect the step-change as a momentary change in an amplitude of the temporal variation as embedded in the measurement signal.

In some embodiments, the processor circuitry is configured to: generate, based on the measurement signal, a monitoring signal that represents momentary change of the weight of the container; and detect the step-change based on the monitoring signal.

In some embodiments, the processor circuitry is configured to generate the monitoring signal to represent a derivative of the measurement signal.

In some embodiments, the processor circuitry is configured to generate a difference signal by subtracting a representation of the temporal variation from the monitoring signal and detect the step-change in the difference signal.

In some embodiments, the processor circuitry is configured to process the difference signal for detection of the temporal variation, wherein the step-change is detected by presence of the temporal variation in the difference signal.

In some embodiments, the temporal variation comprises a minimum flow rate and a maximum flow rate.

In some embodiments, a difference between the minimum flow rate and the maximum flow rate is at least twice an average flow rate generated by the pumping device when operated at the known setting.

In some embodiments, the temporal variation is a sinusoidal variation.

In some embodiments, the temporal variation results in a sequence of time-separated pulses of increased flow rate.

In some embodiments, the temporal variation corresponds to an intermittent activation of the pumping device.

In some embodiments, the fluid in the container is fresh dialysis fluid for use in the extracorporeal treatment of blood in the dialyzer, or water that is mixed with one or more concentrates in the first fluid path to form the fresh dialysis fluid, and wherein the first dialyzer port is an inlet port for the fresh dialysis fluid.

In some embodiments, the fluid in the container is spent dialysis fluid resulting from the extracorporeal treatment of blood in the dialyzer, and the second dialyzer port is an outlet port for the spent dialysis fluid.

In some embodiments, the processor circuitry is further configured to: generate control signals for the pumping device, the adjustment arrangement, and the further pumping device.

In some embodiments, the ultrafiltration parameter is further determined based on fluid flow data representing fluid flow generated in the level adjustment period by one or more other pumping devices in direct or indirect fluid communication with blood in an extracorporeal blood circuit connected to the dialyzer.

A second aspect is a device for controlling flow rate in a system for extracorporeal treatment of blood. The device comprises an input interface, which is configured to receive a measurement signal representative of a momentary weight of a container in the system, the container being connected on a first fluid path to a first dialyzer port for dialysis fluid on a dialyzer for extracorporeal treatment of blood, a pumping device being arranged in the first fluid path, the container being further connected on a second fluid path to an adjustment arrangement for level adjustment of a fluid in the container. The device further comprises processor circuitry, which is connected to the input interface and configured to perform a control procedure in relation to a level adjustment period, in which the adjustment arrangement is selectively activated to perform said level adjustment and the pumping device is operated at a known setting. The processor circuitry, in the control procedure, is configured to, during the level adjustment period, process the measurement signal for detection of a step-change, and initiate a dedicated action upon detection of the step-change.

A third aspect is a system for extracorporeal blood treatment. The system comprises: a dialyzer comprising first and second dialyzer ports for dialysis fluid; a container connected on a first fluid path to the first dialyzer port on the dialyzer; a pumping device in the first fluid path; an adjustment arrangement for level adjustment of a fluid in the container, the adjustment arrangement being connected to the container on a second fluid path; a weighing device arranged to generate a measurement signal representative of a momentary weight of the container; and a device of the second aspect.

A fourth aspect is a computer-implemented method of monitoring ultrafiltration in a system for extracorporeal treatment of blood. The method comprises: receiving a measurement signal representative of a momentary weight of a container in the system, the container being connected on a first fluid path to a first dialyzer port for dialysis fluid on a dialyzer for extracorporeal treatment of blood, a pumping device being arranged in the first fluid path, the container being further connected on a second fluid path to an adjustment arrangement for level adjustment of a fluid in the container; and performing a monitoring procedure in relation to a level adjustment period, in which the adjustment arrangement is selectively activated to perform said level adjustment and the pumping device is operated at a known setting. The monitoring procedure comprises: determining, during a first measurement period prior to the level adjustment period and based on the measurement signal, a first value of a pumping parameter of the pumping device while the adjustment arrangement is deactivated and the pumping device is operated at the known setting; determining, during a second measurement period subsequent to the level adjustment period and based on the measurement signal, a second value of the pumping parameter while the adjustment arrangement is deactivated and the pumping device is operated at the known setting; estimating, based on the first and second values, a time profile of the pumping parameter in the level adjustment period; and determining an ultrafiltration parameter for the level adjustment period based on the time profile.

A fifth aspect is a computer-implemented method of controlling flow rate in a system for extracorporeal treatment of blood. The method comprises: receiving a measurement signal representative of a momentary weight of a container in the system, the container being connected on a first fluid path to a first dialyzer port for dialysis fluid on a dialyzer for extracorporeal treatment of blood, a pumping device being arranged in the first fluid path, the container being further connected on a second fluid path to an adjustment arrangement for level adjustment of a fluid in the container; and performing a control procedure in relation to a level adjustment period, in which the adjustment arrangement is selectively activated to perform said level adjustment and the pumping device is operated at a known setting. The control procedure comprises: processing, during the level adjustment period, the measurement signal for detection of a step-change; and initiating a dedicated action upon detection of the step-change.

A sixth aspect is a computer-readable medium comprising program instructions, which when executed by a processor causes the processor to perform the method of the fourth or fifth aspect.

Embodiments of the second to sixth aspects may correspond to the above-identified embodiments of the first aspect.

Still other objectives, aspects and embodiments, as well as other features and advantages, may appear from the following detailed description, from the attached claims as well as from the drawings.

### BRIEF DESCRIPTION OF THE DRAWINGS

FIG. 1 is a diagram of an example hemodialysis system.

FIGS. 2A-2B are example graphs of flow rate generated by a first pump and a second pump, respectively, in the system of FIG. 1 during a level adjustment period.

FIG. 3 is a flowchart of an example monitoring method performed in the system of FIG. 1.

FIGS. 4A-4B are example graphs of weight signals for containers in the system of FIG. 1 in a time period comprising a level adjustment.

FIGS. 5A-5B are example graphs of irregular flow rates generated by a first pump in the system of FIG. 1 during a level adjustment period.

FIG. 6 is a flowchart of an example control method performed in the system of FIG. 1.

FIGS. 7A-7B are example graphs of a weight signal and its derivative during a time period with a step-change in pumping rate.

FIGS. 8A-8B show examples of temporal variations in flow rate generated by a pump in the system of FIG. 1.

FIGS. 9A-9B are example graphs of a weight signal and its derivative during a time period with a step-change in a fluid flow having a sinusoidal variation.

FIG. 10 is an enlarged view of the derivative weight signal in FIG. 9B.

FIGS. 11A-11B are example graphs of a weight signal and its derivative during a time period with a step-change in a pulsated fluid flow.

FIGS. 12A-12B are flowcharts of example procedures included in the method of FIG. 6.

FIG. 13 is a graph of a difference signal generated from the derivative weight signal in FIG. 9B.

FIGS. 14A-14B correspond to FIGS. 11A-11B but are generated for a different time profile of the level adjustment.

FIG. 15 is a block diagram of an example monitoring device for use in the system of FIG. 1

### DETAILED DESCRIPTION OF EXAMPLE EMBODIMENTS

Embodiments will now be described more fully hereinafter with reference to the accompanying drawings, in which some, but not all, embodiments are shown. Indeed, the subject of the present disclosure may be embodied in many different forms and should not be construed as limited to the embodiments set forth herein; rather, these embodiments are provided so that this disclosure may satisfy applicable legal requirements. Like numbers refer to like elements throughout.

Also, it will be understood that, where possible, any of the advantages, features, functions, devices, and/or operational aspects of any of the embodiments described and/or contemplated herein may be included in any of the other embodiments described and/or contemplated herein, and/or vice versa. In addition, where possible, any terms expressed in the singular form herein are meant to also include the plural form and/or vice versa, unless explicitly stated otherwise. As used herein, “at least one” shall mean “one or more” and these phrases are intended to be interchangeable. Accordingly, the terms “a” and/or “an” shall mean “at least one” or “one or more,” even though the phrase “one or more” or “at least one” is also used herein. As used herein, except where the context requires otherwise owing to express language or necessary implication, the word “comprise” or variations such as “comprises” or “comprising” is used in an inclusive sense, that is, to specify the presence of the stated features but not to preclude the presence or addition of further features in various embodiments.

It will furthermore be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing the scope of the present disclosure. As used herein, the terms “multiple”, “plural” and “plurality” are intended to imply provision of two or more elements. The term “and/or” includes any and all combinations of one or more of the associated listed elements.

Well-known functions or structures may not be described in detail for brevity and/or clarity. Unless otherwise defined, all terms (including technical and scientific terms) used herein have the same meaning as commonly understood by one of ordinary skill in the art to which this disclosure belongs.

The present disclosure relates to a technique of monitoring a fluid flow out of a container, based on the weight of the container, while there is a concurrent and more or less unknown fluid flow into the container. Correspondingly, the technique is equally applicable for monitoring the fluid flow into a container while there is a concurrent and more or less unknown fluid flow out of the container. The technique is specifically adapted for use in systems for extracorporeal (EC) blood treatment, to estimate the fluid flow of fresh dialysis fluid into the system and/or the fluid flow of spent dialysis fluid from the system, for the purpose of monitoring ultrafiltration of blood in the system. As noted in the Background section, machines for EC blood treatment may be configured to monitor and control ultrafiltration based on signals from scales, on which containers for fresh and spent dialysis fluid are releasably arranged. While such machines are commonly used for so-called acute treatment, the technique is not limited to any specific type of EC blood treatment. The technique enables uninterrupted blood treatment by allowing the machine to intermittently replenish fluid in a container while pumping fluid from the container to the system, and/or to intermittently drain fluid from a container while pumping fluid from the system to the container.

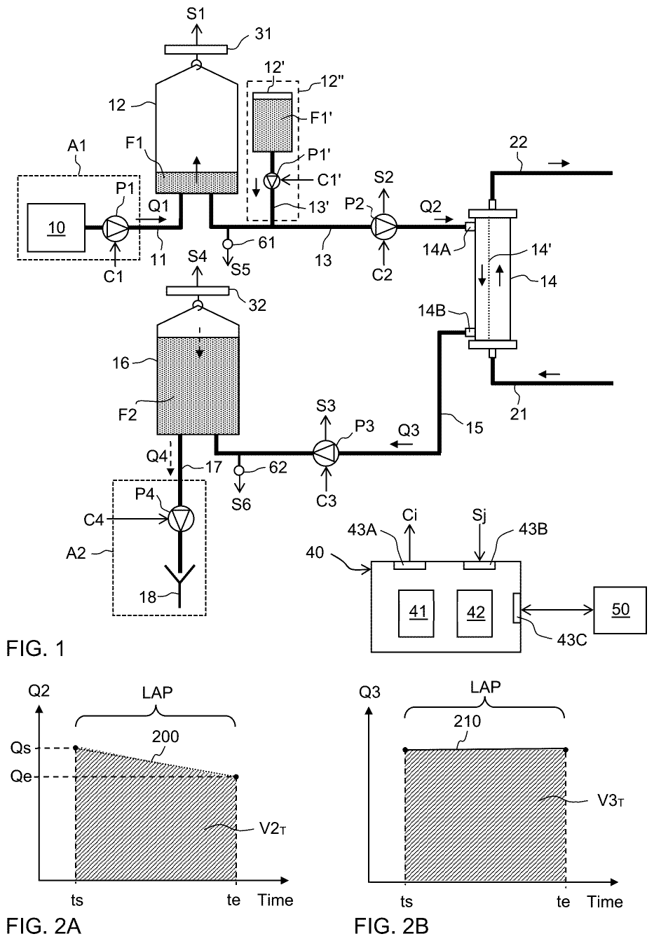

The technique will be exemplified with reference to an example system for EC blood treatment depicted in FIG. 1. The system may (but need not) be implemented by a machine for treatment of AKI (“acute dialysis”). In the illustrated example, the system is configured for hemodialysis (HD) treatment. The system comprises a replenishment arrangement A1, which comprises a fluid source 10 and a fluid pump (“pumping device”) P1. The pump P1 is operable to pump a first fluid F1 from the source 10 through a connecting line or tube (“fluid path”) 11 to a first container 12. The first container 12 may be flexible or rigid and is arranged to receive the first fluid F1. Depending on implementation, as described further below, the first fluid F1 may be fresh dialysis fluid or water. The source 10 may be a stand-alone unit at the point of care or a centralized unit for fluid supply to a plurality of systems. If F1 is fresh dialysis fluid, the source 10 may be a conventional device for preparation of dialysis fluid. If F1 is water, the source 10 may be a conventional device for preparation of purified water, for example by reverse osmosis. The replenishment arrangement A1 is also denoted “level adjustment arrangement” since is it operable to selectively increase the level of the fluid F1 in the container 12.

The container 12 is connected on a connecting line or tube (“fluid path”) 13 to an inlet port 14A of a dialyzer 14. The connecting line 13 is also denoted “supply line” in the following. A fluid pump P2 is operable to pump the fluid F1 from the container 12 through the supply line 13 to the dialyzer 14. The dialyzer 14 is a blood processing unit and may be of any conventional type. The dialyzer 14 comprises a semipermeable membrane 14′ which separates the dialyzer 14 into a first chamber for dialysis fluid and a second chamber for blood. The first chamber has an inlet port 14A for fresh dialysis fluid and an outlet port 14B for spent dialysis fluid. Correspondingly, the second chamber has first and second ports for connection to blood lines 21, 22 carrying incoming and outgoing blood, respectively. The blood lines 21, 22 and the second chamber of the dialyzer 14 are thereby included in an extracorporeal blood circuit. The blood is typically extracted from the circulatory system of a patient, pumped through the dialyzer 14 and returned to the circulatory system of the patient. As is well-known to the skilled person, water and substances may be exchanged between the blood and the dialysis fluid across the membrane 14, to thereby treat the blood. The spent dialysis fluid, also known as “effluent”, contains excess water and waste removed from the blood. The excess water is commonly known as “ultrafiltrate” and the process of removing the excess water is known as “ultrafiltration”.

The outlet port 14B of the dialyzer 14 is connected on a connecting line or tube (“fluid path”) 15 to a second container 16. The second container 16 may be flexible or rigid and is arranged to hold a second fluid F2, which is the above-mentioned spent dialysis fluid or effluent. A fluid pump P3 is operable to pump the fluid F2 from the dialyzer 14 through the connecting line 15 to the container 16. The connecting line 15 is also denoted “effluent line” in the following. The container is connected on a connecting line or tube (“fluid path”) 17 to a draining arrangement A2, which comprises a drain or receptacle 18 and a fluid pump P4. The connecting line 17 is also denoted “drain line” in the following. The pump P4 is operable to pump the second fluid F2 through the drain line 17 into the drain 18. The draining arrangement A2 is also denoted “level adjustment arrangement” since is it operable to selectively decrease the level of the fluid F2 in the container 16.

In the example of FIG. 1, the first container 12 is hung from a first scale 31, and the second container 12 is hung from a second scale 32. The scales 31, 32 are configured to measure the weight of the respective container 12, 16. In a variant, the respective container may be placed to rest on the scale.

In some embodiments, as shown in FIG. 1, the system may comprise one or more pressure sensors 61, 62, which are configured to measure fluid pressure. In the illustrated example, pressure sensor 61 is arranged to measure fluid pressure in the supply line 13 upstream of pump P2, and pressure sensor 62 is arranged to measure fluid pressure in the effluent line 15 downstream of pump P3. As will be described in more detail below, the pressure sensors 61, 62 are thereby responsive to varying pressure resulting from variation in the level of fluid in the respective container 12, 16.

If the first fluid F1 is water, the system also comprises one or more concentrate supply arrangements 12″, each of which being arranged to meter a liquid concentrate F1′ into the supply line 13 so as to form the fresh dialysis fluid in the supply line 13. In the example of FIG. 1, the concentrate supply arrangement 12″ comprises a container 12′ holding a liquid concentrate F1′, a connecting line 13′ in fluid communication with the supply line 13, and a fluid pump P1′ which is operable to pump the liquid concentrate through the connecting line 13′ into the supply line 13. The concentrate supply arrangement(s) 12″ may be connected to the supply line 13 upstream (as shown) or downstream of the pump P2.

The system may be configured by combining one or more disposables with a machine for EC blood treatment. The machine may comprise the scales 31, 32, the pumps P1′, P2 and P3, and the pressure sensors 61, 62, as well as other conventional components well-known to the skilled person. The pumps P1′, P2, P3 may or may not be peristaltic pumps. The level adjustment arrangements A1, A2 may also be part of the machine. The one or more disposables may include the containers 12, 16, the connecting lines 13, 15, the dialyzer 14, and the blood lines 21, 22. The connecting lines 11, 17 may also be part of the disposable(s). The concentrate container(s) 12′ and the connecting line(s) 13′ may be part of the machine or the disposable(s). The skilled person understands that the system may include further conventional components, which may be part of the machine or the disposable(s).

A control device 40 is configured to control the operation of the system by use of control signals Ci, which are output on a first signal interface 43A. The control device 40 may or may not be integrated in the machine for EC blood treatment. In the example of FIG. 1, the control signals Ci include control signals C1′, C1-C4 for the pumps P1′, P1-P4. The control device 40 operates the system to perform blood treatment in accordance with a control program comprising computer instructions. The control program is configured to operate based on one or more input signals Sj, which are received on a second signal interface 43B. User inputs may be entered by a user via a human-machine interface (HMI) 50, which in connected to a third interface 43C of the control device 40. The HMI 50 may comprise any data entry equipment, such as a keyboard, keypad, control button(s), touch screen, computer mouse, track pad, microphone, camera, etc. The HMI 50 may also comprise any data feedback equipment, such as a display, speaker, indicator lamps, alarm device, etc. In the example of FIG. 1, the input signals Sj comprise a measurement signal S1 (“weight signal”) generated by the scale 31 to represent the momentary weight of the container 12, signals S2, S3 (“speed signals”) generated by the pumps P2, P3 to represent the momentary pumping speed of the respective pump, a measurement signal S4 (“weight signal”) generated by the scale 32 to represent the momentary weight of the container 16, and measurement signals S5, S6 (“pressure signals”) generated by the pressure sensors 61, 62 to represent fluid pressure. It should also be noted that all of the input signals S1-S6 may not be used for controlling the operation of the system. As described below, at least some of the input signals S1-S6 may be used for monitoring and quantifying ultrafiltration performed by the system.

The control device 40 comprises processor circuitry 41 and computer memory 42. The above-mentioned control program is stored in the memory 42 and executed by the processor circuitry 41, which comprises one or more processors of suitable type. The control program may be supplied to the control device 40 on a computer-readable medium, which may be a tangible (non-transitory) product (e.g., magnetic medium, optical disk, read-only memory, flash memory, etc.) or a propagating signal. It may be noted that two or more of the interfaces 43A-43C may be combined into one physical unit.

The operation of the system in FIG. 1 will be briefly described under the assumption that the fluid F1 is fresh dialysis fluid. The pump P2 is continuously operated to supply fresh dialysis fluid F1 from the container 12 at a flow rate Q2, which may be fixed or variable. The fluid passes the first chamber of the dialyzer 14, as shown by a downward arrow, while interacting via the semipermeable membrane 14′ with the blood that is concurrently pumped through the second chamber of the dialyzer 14 (as shown by an upward arrow). The pump P3 is continuously operated to draw the spent dialysis fluid F2 from the dialyzer 14 at a flow rate Q3, which may be fixed or variable. The pumping speeds of the pumps P2, P3 are set to achieve target values of Q2 and Q3. To achieve a high accuracy of the flow rates Q2, Q3, the pumps P2, P3 are controlled (by control signals C2, C3) based on the momentary flow rate that is given by the signals S1, S4. The momentary flow rate corresponds to the rate of weight change of the respective container 12, 16. The difference between Q2 and Q3 defines the ultrafiltration rate (“UF rate”), which is the rate at which ultrafiltrate is drawn from the 5 blood in the dialyzer 14. The total amount of ultrafiltrate (“total UF”) during a treatment session corresponds to the integrated difference between Q2 and Q3 over time and may be calculated in various ways (below). The control device 40 is configured to operate the pumps P2, P3 to achieve a target value of the total UF and/or a predefined time profile of the UF rate during the treatment session.

Intermittently during the treatment session, when the container 12 is deemed to be sufficiently depleted of fresh dialysis fluid F1, the replenishment arrangement A1 is activated to pump fresh dialysis fluid F1 into the container 12, at flow rate Q1, to raise the level in the container 12, as indicated by a solid arrow. The need to replenish the container 12 may be determined based on the weight of the container 12 given by signal Si. Similarly, when the container 16 deemed to be sufficiently full, the draining arrangement A2 is activated to pump the spent dialysis fluid F2 from the container 16 to the drain 18, at flow rate Q4, to decrease level in the container 16, as indicated by a dashed arrow. The refilling and draining of the containers 12, 16 may be performed concurrently or independently of each other. It may be noted that, if the respective container 12, 16 is made of flexible material, the level of fluid in the container 12, 16 may not increase/decrease linearly if the container is deformed by the inflow/outflow of fluid.

The operation of the system in FIG. 1 is similar when the fluid F1 is water. The main difference is that the one or more concentrate supply arrangements 12″ are operated concurrently with the pump P2 to pump concentrate(s) F1′ into the supply line 13. In the example that the system includes a single supply arrangement 12″, as shown, the pump P2 is operated to pump water from the container 12, and the pump P1′ is operated at a speed with a predefined relation to the speed of the pump P2 to achieve a predefined dilution of the concentrate F1′ by the water F1. The predefined relation may be given by calibration, for example to achieve a target conductivity of the resulting fresh dialysis fluid as measured by a sensor (not shown). Given the predefined relation, the speed of the pump P2 is controlled based on the rate of weight change of the container 12, given by signal S1, to achieve a target value of the flow rate Q2 of fresh dialysis fluid into the dialyzer 14. Otherwise, the operation of the system is the same as when the fluid F1 is fresh dialysis fluid as described above.

It may be noted that the flow rates Q1, Q4 may be set as high as possible to achieve fast replenishment and drainage of the respective container 12, 16. To achieve high flowrates Q1, Q4 at low cost, the pumps P1 and P4 may be of a construction that provides a comparatively low accuracy of the resulting flow rate. Further, for cost reasons, it may be undesirable to include high accuracy flow meters to quantify Q1 and Q4. In this situation, when Q1 and Q4 are largely unknown, the signals S1 and S4 cannot be reliably used to control the pumps P2, P3 to achieve Q2 and Q3. Thus, with respect to UF monitoring, the control device 40 is “blind” when the either of the arrangements A1, A2 is operated to adjust the level in the container 12 or 16. In the following, the time period when A1 or A2 is activated is denoted a “level adjustment period”, LAP. One approach to address the “blindness” is to stop the treatment during each LAP, by at least stopping the pumps P2, P3. This approach reduces the efficacy of the treatment. Another approach is to continue the treatment during the LAP by operating the pumps P2, P3 with fixed settings and calculate the total UF for the LAP by use of predefined nominal values for the resulting flow rates Q2, Q3 during the LAP. This approach introduces an uncertainty into the calculation of the total UF. Even at fixed settings, the flow rates Q2, Q3 may exhibit minor changes over time, for example as a result of wear in the pumps, changing conditions at the pumps, etc.

Some embodiments of the present disclosure aim at providing information about the UF rate during the LAP. In some embodiments, if the LAP relates to the container 12, this is achieved by measuring the flow rate Q2 before the LAP and after the LAP, by use of the weight signal S1, and estimate the time profile of the flow rate Q2 during the LAP based on the measured flow rates. As used herein, “time profile” refers to a mapping of a quantity over time. Correspondingly, if the LAP relates to the container 16, the flow rate Q3 may be measured before the LAP and after the LAP, by use of the weight signal S4, and the time profile of the flow rate Q3 during the LAP may be estimated based on the measured flow rates. Based on the estimated time profile of Q2 and/or the estimated time profile of Q3, the ultrafiltration during the LAP may be quantified. The uncertainty in ultrafiltration during LAP is reduced by the measurement of actual flow rates before and after the LAP.

The foregoing concept is illustrated in FIG. 2A, which is a graph of Q2 as a function of time during an LAP resulting in a replenishment of the container 12. As shown, a start flow rate Qs is measured at a time ts before the LAP, and an end flow rate Qe is measured at a time te after the LAP. By using Qs and Qe as reference values, it is possible to estimate the time profile of the fluid flow into the dialyzer 14 during the LAP. In the illustrated example, the estimated time profile 200 is given by assuming a linear change in flow rate between Qs and Qe. The hatched area in FIG. 2A corresponds to the total amount (V2_{T}) of fresh dialysis fluid that is pumped into the dialyzer 14 during the LAP. FIG. 2B corresponds to FIG. 2A and shows Q3 as a function of time during the replenishment of the container 12 in FIG. 2A. Here, it is assumed that the container 16 is not drained during the replenishment of the container 12 and that the flow rate Q3 is repeatedly measured by use of the weight signal S4 during the LAP, resulting in the measured flow profile 210. The hatched area in FIG. 2B corresponds to the total amount (V3_{T}) of spent dialysis fluid that is pumped out of the dialyzer 14 during the LAP. The difference between V3_{T} and V2_{T} provides the estimated total UF during the LAP, and the difference between the flow profiles 200 and 210 provide an estimated time profile of the UF rate during the LAP.

It is realized that Q2 and Q3 may be switched between FIG. 2A and FIG. 2B, so that an estimated time profile of Q3 is obtained based on Qs and Qe during drainage of the container 16, and that a measured time profile of Q2 is obtained based on the weight signal S i during the drainage of the container 16 provided that the container 12 is not replenished during the drainage of the container 16.

The principle embodied in FIGS. 2A-2B is equally applicable if the replenishment of the container 12 overlaps in time with the drainage of the container 16. In this case, the flow profile 210 is at least partly replaced for an estimated flow profile, given by a start and end flow rates of Q3, which are measured before and after the drainage of the container 16.

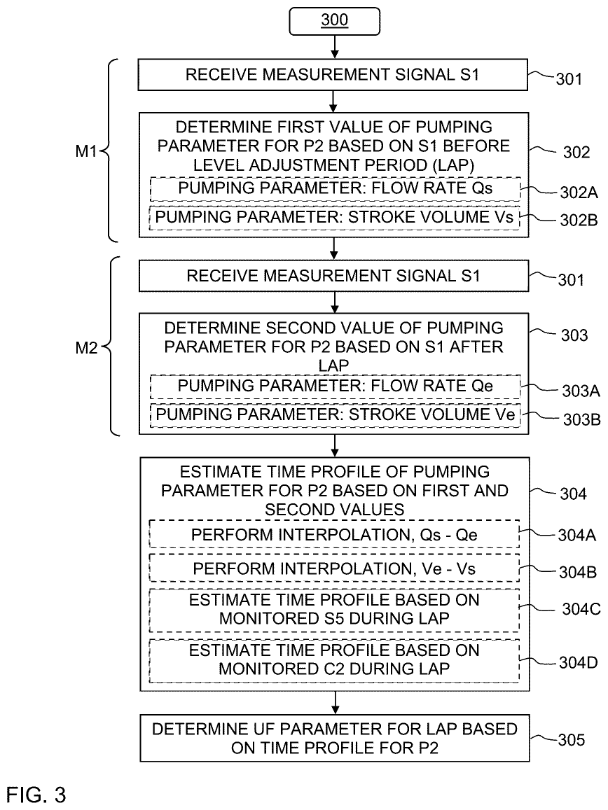

FIG. 3 is a flowchart of an example method 300 which may be performed by the control device 40 in the system of FIG. 1. For simplicity, the method 300 will be described under the assumption that the container 12 is to be replenished whereas the container 16 is not to be drained. In other words, A1 is activated during the LAP and A2 is not.

Steps 301 and 302 correspond to a first measurement period, M1, before the LAP. In step 301, the weight signal S1 generated by the scale 31 is received. The weight signal S1 represents the momentary weight of the container 12 and comprises a time series of momentary weight values. In this context, “momentary” may be on any time scale that is relevant for the subsequent processing in step 302. For example, S1 may have a time resolution on the order of milliseconds or seconds. Step 302 comprises determining, based on S1 as received during M1, a first value of a pumping parameter of P2, which is operated at a known setting during M1. During M1, A1 is deactivated. In some embodiments, as indicated by step 302A, the pumping parameter is or represents flow rate, specifically Q2 generated by P2. The flow rate Q2 may be determined based on AW/At, with AW being a change in weight, given by S1, over a time period At. As used herein, “flow rate” may be given in terms of weight (mass) or volume. Thus, AW/At may or may not be divided by the density of the fluid in the container, be it water or fresh dialysis fluid, to give the flow rate Q2. In the example of FIG. 2A, the first value corresponds to Qs.

Although not shown in FIG. 3, after step 302, the system is controlled to perform the LAP, by activating A1. During the LAP, P2 is operated at the known setting.

After completion of the LAP, when the container 12 is sufficiently replenished with fluid and A1 has been deactivated, the method performs steps 301 and 303 in a second measurement period, M2. In step 301, the weight signal S1 generated by the scale 31 is received. Step 303 comprises determining, based on S1 as received during M2, a second value of a pumping parameter of P2, which is operated at the known setting during M2. Assuming that the pumping parameter is flow rate, as indicated by step 303A, the second value is determined in the same way as in step 302A, based on ΔW/Δt. In the example of FIG. 2A, the second value corresponds to Qe.

In the following description it is assumed that the “known setting” of the pump P2, which is maintained throughout M1, LAP and M2, is a fixed setting of the pump P2. At the fixed setting, the pump P2 is assumed to operate at a fixed pumping speed. The fixed setting may be a predefined setting for the method 300. Alternatively, the fixed setting may be given by a current setting of the pump P2 when M1 is initiated. The use of the fixed setting will generally facilitate steps 304-305 (below). However, it is conceivable that the known setting corresponds to a predefined variation in the pumping speed during M1, LAP and M2. It is also to be noted that step 301 need not be performed only during M1 and M2, but may be performed also during the LAP, for example to enable the method of FIG. 6 or certain embodiments of step 305 (below).

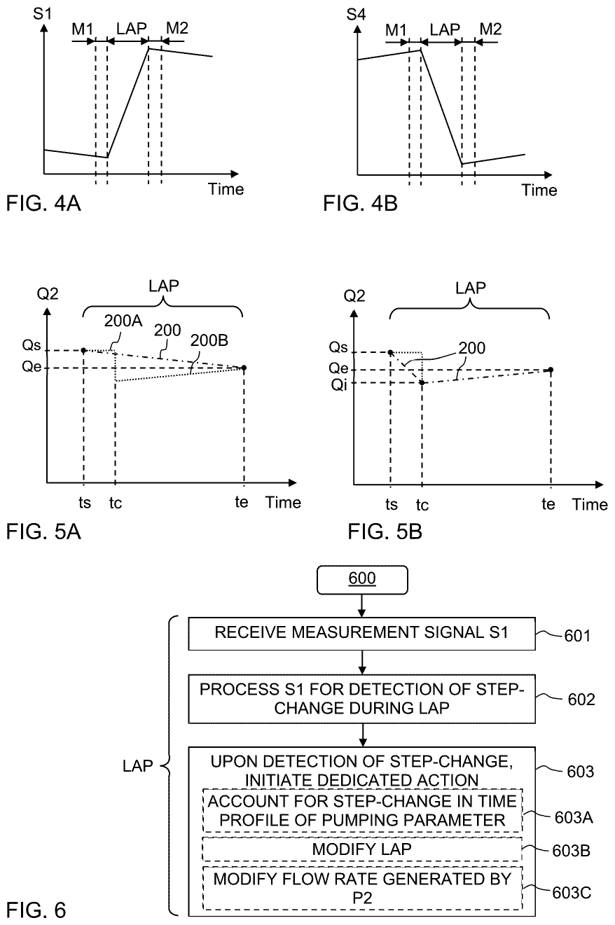

FIG. 4A is a graph of an example weight signal S1 obtained during M1, LAP and M2 while the pumping speed of P2 is fixed. As seen, the weight of the container 12 decreases before LAP as a result of the pumping action of P2, increases rapidly during LAP when A1 is activated to refill the container 12, and then decreases after LAP as a result of the pumping action of P2. During LAP, the change in weight is the combined result of the refilling by A1 and the pumping action by P2.

Reverting to the method 300 in FIG. 3, step 304 is performed after step 303 to estimate a time profile of the fluid flow Q2 generated by the pump P2 during the LAP, based on the first and second values determined by steps 302, 303. This time profile is denoted TPF2 in the following. Assuming that the first and second values correspond to Qs and Qe in FIG. 2A, step 304 may determine TPF2 by interpolating Qs and Qe by use of any interpolation function. In some embodiments, the interpolation function is linear, resulting the time profile 200 in FIG. 2A. The use of a linear interpolation function is simple and likely to be reasonably accurate, at least in the absence of step-changes in Q2 during the LAP (FIGS. 5A-5B, below).

Step 305 is performed after step 304 and comprises determining an ultrafiltration (UF) parameter for the LAP based on TPF2, as estimated in step 304A. In the HD system shown in FIG. 1, the UF parameter is further determined based on fluid flow data (FFD) that represents the flow rate Q3 generated by the pump P3 during the LAP. Assuming that A2 is deactivated during the LAP, the FFD is directly or indirectly given by the weight signal S4 from the scale 32. The FFD may take different forms, depending on the UF parameter to be determined. In one example, the FFD designates the total amount of fluid pumped by pump P3 during LAP (cf. V3_{T} in FIG. 2B) and is given by the difference in weight of the container 16 between the start and end of the LAP. In another example, the FFD is a time profile for Q3 (cf. 210 in FIG. 2B), which is given by step-wise weight changes of the container 16 during the LAP and may be calculated as a derivative of the weight signal S4. In another example, assuming that A2 is also activated during the LAP, the FFD may be determined by performing steps 301-304 in relation to the container 16 (below).

Depending on implementation, the UF parameter may take different forms. In a first example, the UF parameter designates the total amount of ultrafiltrate extracted from blood in the dialyzer 14 during the LAP. Such a UF parameter is given by the difference between V3_{T} and V2_{T} in FIGS. 2A-2B. In a second example, the UF parameter is a deviation of the total amount of ultrafiltrate from a target value. In a third example, the UF parameter is a time profile of UF rate during the LAP and is given by the difference between TPF2, given by step 304, and a time profile of Q3, given by the FFD. In a fourth example, the UF parameter is a time profile representing the deviation of the UF rate during the LAP from a target time profile.

It may be noted that TPF2 may be inherent to the calculations in steps 304-305. For example, V2_{T} may be calculated by assuming a certain TPF2 and does not require TPF2 to be explicitly derived. For example, by assuming that TPF2 is linear, V2_{T} may be directly calculated as ΔLAP·(Qs+Qe)/2), with ΔLAP being the duration of the LAP.

It should also be noted that the pumping parameter need not designate the flow rate of the pump P2. In an alternative, the pumping parameter represents the stroke volume of the pump P2. This presumes that the pump P2 is a positive displacement pump, for example a peristaltic pump as commonly used in systems for EC blood treatment. Thus, the first value is a measured stroke volume (Vs) of the pump P2 during M1 (cf. step 302B), and the second value is a measured stroke volume (Ve) of the pump P2 during M2 (cf. step 303B).

The stroke volume may be determined in steps 302B and 303B based on the signals S1 and S2 as obtained during M1 and M2. The signal S2 represents the pumping speed of the pump P2 and thereby generally designates the number of strokes performed by the pump P2 per unit time. The stroke volume may be calculated by relating a measured weight change (given by Si) to a measured pumping speed (given by S2) for a given time period. As used herein, “stroke volume” may be given in terms of volume or weight. The signal S2 may take many different forms. In some embodiments, the signal S2 comprises a predefined number of pulses for each pumping stroke of the pump P2. For example, the pulses may originate from an encoder in the pump P2, a step count signal from a stepper motor in the pump P2, etc. In a variant, the signal S2 is omitted and the pumping speed in M1 and M2 is a nominal value associated with the known setting of the pump P2 during M1 and M2, respectively. If the known setting is a fixed setting, the nominal value is the same in M1 and M2.

In step 304, as indicated by step 304B, Vs and Ve may be interpolated by use of any interpolation function, for example a linear function, to determine an estimated time profile for the stroke volume (“stroke volume profile”, TPV2) during the LAP.

Reverting to step 305, TPV2 may be processed in diverse ways for determination of the UF parameter. Step 305 may comprise determining a fluid flow parameter for the LAP based on TPV2 and based on the signal S2 during the LAP, and determining the UF parameter based on the fluid flow parameter. For example, the fluid flow parameter may be TPF2 or V2_{T}. The use of S2 may improve the accuracy of the UF parameter since S2 represents the actual performance of P2 in terms of pumping speed. For example, stalling events or speed fluctuations of P2 during the LAP will be automatically accounted for and included in the fluid flow parameter. Step 305 may determine TPF2 by multiplying individual values in TPV2 by corresponding individual values of the pumping speed of the pump P2 during the LAP, given by S2. Step 305 may calculate V2_{T} by determining, based on S2, a count (NS) of the pumping strokes performed by P2 during the LAP, calculating an average stroke volume (Vavg) for the LAP based on TPV2, and determining V2_{T} as a function of NS and Vavg, for example as (Vavg·NS). If the stroke volume profile is assumed to be linear, Vavg for the LAP may be calculated as (Vs+Ve)/2. As an alternative to be determined from the signal S2, NS may be a nominal value given by the known setting of P2 during the LAP, or an estimated value determined based on the measured pumping speeds in M1 and M2.

A further variant of estimating the time profile of the pumping parameter is represented by step 304C, in which the pumping parameter may be either flow rate or stroke volume of the pump P2. Reverting to FIG. 1, the pressure signal S5 represents the pressure at the inlet to the pump P2. This pressure will vary slightly with the level of fluid in the container 12, since the level will change the hydrostatic pressure at the inlet to the pump P2. This is true for any placement of the container 12 in relation to the pump P2. For example, the hydrostatic pressure may change on the order of 2-50 mmHg as the container 12 is refilled during the LAP. It is well-known that changes in the inlet pressure of a pump may affect its stroke volume and thereby affect the flow rate generated by the pump. The Applicant has realized that this potential source of inaccuracy may actually be used to determine the time profile of the pumping parameter, if the inlet pressure of pump P2 is measured during the LAP. Step 304C presumes access to calibration data that relates inlet pressure to flow rate or stroke volume of the pump P2. In principle, the pressure signal S5 may be directly converted into the time profile by use of the calibration data. Often, the calibration data is not sufficiency accurate to enable such a direct conversion. The pressure signal S5 may instead be converted into the time profile by fitting a curve of pumping parameter values, which is given by the pressure signal S5 and the calibration data, to the first and second values measured by steps 302 and 303.

The calibration data may be predefined and stored in the internal memory of the control device (cf. 42 in FIG. 1). Alternatively, the calibration data may be determined while the container 12 is being gradually depleted before an LAP, by relating measured inlet pressure (given by S5) to measured weight change (given by S1), optionally in combination with measured pumping speed (given by S2). In some embodiments, the calibration data is represented as a proportionality factor between inlet pressure and flow rate, or between inlet pressure and stroke volume. In other embodiments, the calibration data defines a non-linear dependence and may be given by a function or a look-up table.

Another variant of estimating the time profile of the pumping parameter is represented by step 304D, in which the pumping parameter may be either flow rate or stroke volume of the pump P2. Step 340D may be performed by analogy with step 340C, while using the control signal C2 for the pump P2 instead of the pressure signal S5. The rationale for step 340D is that the control signal C2 will be generated based on the momentary flow rate given by the weight signal S1, as explained hereinabove. This means that the control signal C2 will automatically account for the impact of the fluid level in the container 12 on the hydrostatic pressure at the inlet to the pump P2. Thus, it is possible to derive calibration data that relates control values of the signal C2 to flow rate or stroke volume of the pump P2. By use of such calibration data, the control values may be converted into a time profile of the pumping parameter. By analogy with step 304C, the calibration data may be predefined or determined while the container 12 is being gradually depleted before an LAP by relating control signal values (given by C2) to measured weight change (given by Si), optionally in combination with measured pumping speed (given by S2).

Although the method 300 has been described with reference to a refilling operation of the container 12 performed by A1, it is equally applicable to a draining operation of the container 16 performed by A2. FIG. 4B is a graph of an example weight signal S4 obtained during M1, LAP and M2. As seen, the weight of the container 16 increases before LAP as a result of the pumping action of the pump P3, decreases rapidly during LAP when A2 is activated to drain the container 16, and then increases after LAP as a result of the pumping action of the pump P3. When the method 300 is applied for monitoring in relation to the container 16, step 301 receives the weight signal S4, and steps 302-303 determine first and second values for a pumping parameter of the pump P3 based on S4. Step 304 estimates a time profile for the flow rate Q3 (TPF3) or a time profile for the stroke volume of pump P3 (TPV3), based on the first and second values. With reference to step 304C, the time profile may be estimated based on the pressure signal S6, which represents the fluid pressure at the outlet of the pump P3, by analogy with the description hereinabove. With reference to step 304D, the time profile may be estimated based on the control signal C3, by analogy with the description hereinabove. Step 305 then determines the UF parameter based on the estimated TPF3 or TPV3 and further based on FFD that represents the flow rate Q2 generated by the pump P2 during the LAP.

Generally, the method 300 in FIG. 3 is appliable to any system for extracorporeal blood treatment that comprises at least one combination of a level adjustment arrangement, a container and a pumping device. This combination may be arranged to either supply dialysis fluid or receive spent dialysis fluid.

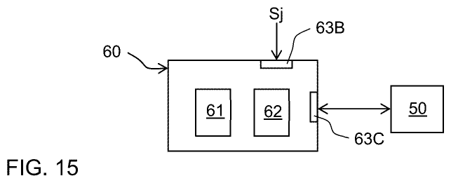

The method 300 need not be performed by the control device 40 (FIG. 1) that controls the operation of the system but may be performed by a physically separate monitoring device 60, shown in FIG. 15, provided that the monitoring device 60 is able to determine suitable time points for performing M1 and M2 in relation to the LAP. Such suitable time points may be signaled to the monitoring device 60 by the control device 40. In the example of FIG. 15, the monitoring device 60 comprises components 61, 62, 63B, 63C that correspond to the components 41, 42, 43B, 43C in the control device 40.

Although the method 300 has been described in relation to a system for hemodialysis (HD), it is equally applicable to other types of blood treatment, including but not limited to hemodiafiltration (HDF) and hemofiltration (HF).

In HDF, a dialysis fluid is directed through the dialyzer, as in HD, and a replacement fluid is delivered directly to the blood in the extracorporeal blood circuit. The replacement fluid is a type of dialysis fluid. The replacement fluid may be added to the blood upstream (pre-infusion) and/or downstream of the dialyzer (post-infusion). In the example of FIG. 1, a dedicated supply line for replacement fluid may be added to extend from the supply line 13, for example at a location downstream of the pump P2, to one or more infusion sites on the blood lines 21, 22. A dedicated pump may be arranged to pump part of the generated dialysis fluid to the infusion site(s). Alternatively or additionally, replacement fluid may be separately pumped to the infusion sites from a dedicated container, which may or may not be arranged on a scale. The replacement fluid in the dedicated container may be produced on demand or be a ready-made fluid. The method 300 in FIG. 3 may be adapted to HDF by modification of step 305 to account for the replacement fluid that is added to the blood during the LAP. If the replacement fluid is diverted from the supply line 13 downstream of the pump P2, no modification of the method 300 is necessary. Otherwise, step 305 is modified to combine the flow of replacement fluid with the fluid flow Q2 when calculating the UF parameter. The flow of replacement fluid may be given by a flow meter, volumetric dosing or a scale. The draining of spent dialysis fluid in an HDF system is the same as in the HD system of FIG. 1. Thus, irrespective of how dialysis fluid is supplied to the HDF system, the fluid flow Q3 may be determined in accordance with the method 300 in FIG. 3 and used for determining the UF parameter.

In HF, replacement fluid is delivered directly to the blood in the extracorporeal blood circuit (pre-infusion and/or post-infusion), fluid is drawn from the blood through the semi-permeable membrane in the dialyzer, and no dialysis fluid is supplied to the dialyzer. In the example of FIG. 1, the dialyzer port 14A may be plugged, and the supply line 13 may instead be connected to one or more infusion sites on the blood lines 21, 22. Alternatively or additionally, replacement fluid may be separately pumped to the infusion sites from a dedicated non-refillable container. The method 300 in FIG. 3 may be adapted to HF by modification of step 305 to account for the replacement fluid that is added to the blood during the LAP. If the replacement fluid is supplied by the pump P2, no modification of the method 300 is necessary. Otherwise, step 305 is modified to replace the fluid flow Q2 with the flow of replacement fluid when calculating the UF parameter. Again, the draining of spent dialysis fluid in an HF system is the same as in the HD system of FIG. 1. Thus, irrespective of how dialysis fluid is supplied to the HDF system, the fluid flow Q3 may be determined in accordance with the method 300 in FIG. 3 and used for determining the UF parameter.

The method 300 in FIG. 3 presumes that there are no sudden, step-like changes in the flow rate Q2 or Q3 during the LAP. Such a step-change may occur as a result of a hardware error in the machine, for example in a pump P2, P3 or a scale 31, 32. The step-change may also be the result of a handling error, for example that one of the containers 12, 16 is moved during the LAP so as to offset the weight measured by the scale 31, 32. A container movement may be caused by it being touched, by one of the connecting lines to the container being stretched, by the machine being moved, etc. Since the pumps P2, P3 are controlled based on the weight signals S1, S4, a sudden change in S1, S4 is likely to disrupt the control of the pumps P2, P3.

If step-changes during the LAP go undetected, the UF parameter determined in step 305 may be erroneous, especially if the time profile of the pumping parameter is estimated by interpolation (cf. steps 304A, 304B). Even if the time profile is estimated based on the pressure signal S5, S6 or the control signal C2, C3 (cf. steps 304C, 304D), these signals have slow response to changes in flow rate and may not properly reproduce step-changes.

The impact of step-changes is illustrated in FIG. 5A, which is graph of flow rate Q2 as a function of time during replenishment of the container 12. Like in FIG. 2A, Qs is measured at a time ts before the LAP, and Qe is measured at a time te after the LAP. Based on Qs and Qe, step 304A may perform a linear interpolation to estimate a time profile 200 for the LAP. The actual flow rate Q2 is indicated by dotted lines in FIG. 5A. A step-change (decrease) in Q2 occurs a time tc, so that the actual time profile of Q2 comprises a first sub-profile 200A, and a second sub-profile 200B, which is offset to the first sub-profile 200A by the step-change. It is realized that V2_{T} determined from the estimated time profile 200 may differ from V2_{T} determined from the actual time profile 200A, 200B, resulting in a corresponding error in the UF parameter determined by step 305. FIG. 5B corresponds to FIG. 5A but assumes that the step-change is detected and quantified at time tc so that an intermediate flow rate Qi is estimated for time tc. Based on Qs, Qi and Qe, a time profile 200 may be estimated that more closely represents the actual time profile and thereby reduces the error in V2_{T}, if determined.

FIG. 6 is a flowchart of an example method 600 for addressing the problem of step-changes during the LAP. The method 600 may be performed by the control device 40 in FIG. 1 or the monitoring device 60 in FIG. 15. The method 600 is based on the insight that it is possible to improve the accuracy of the UF parameter determined in step 305 if step-changes are detectable. Although the method 600 will be described with reference to replenishment of the container 12 in FIG. 1, it is equally applicable to drainage of the container 16.

The method 600 is performed in relation to, and preferably during, an LAP. In the following, it is assumed that the method 600 is performed between M1 and M2 in FIG. 3. In step 601, the weight signal S1 generated by the scale 31 is received. In step 602, the weight signal S1 is processed for detection of a step-change. As used herein, “step-change” in a signal refers to a change that has a magnitude in excess of regular variations of the signal, in this case variations resulting from regular operation of A1 and the pump P2. In some embodiments, step 602 may also quantify the step-change by determining a magnitude of the step-change. In step 603, dedicated action is taken when a step-change is detected in step 602. The dedicated action may differ depending on implementation and may or may not use the magnitude of the step-change.

In a first implementation, represented by step 603A in FIG. 6, the dedicated action comprises accounting for the step-change in the time profile of the pumping parameter when determined by step 304 and thus in the calculation of the UF parameter in step 305. The first implementation presumes that the step-change is quantified in step 602 (cf. Qi in FIG. 5B). The time profile may be determined as described with reference to FIG. 5B, by accounting for the magnitude and timing (cf. tc) of the step-change.

In a second implementation, represented by step 603B in FIG. 6, the dedicated action comprises modifying the LAP based on the step-change. For example, at least one of A1 and the pump P2 may be deactivated. In one embodiment, A1 is deactivated to terminate the LAP while the pump P2 continues to be operated at the known setting. Step 302 may then be performed to obtain a new first value, whereupon A1 is again activated to perform a second LAP. At completion of the second LAP, step 304 may estimate both the time profile for the first LAP and the time profile for the second LAP, based on the first and second values determined by steps 302, 303.

In a third implementation, represented by step 603C in FIG. 6, the dedicated action comprises modifying the pumping speed of the pump P2 based on the step-change to at least partly compensate for the change in flow rate Q2 caused by the step-change. The third implementation presumes that the step-change is quantified in step 602. The pumping speed of the pump P2 may be modified during the LAP or subsequent to the LAP.

It is also conceivable that the dedicated action comprises a combination of two or more of the first, second and third implementations.

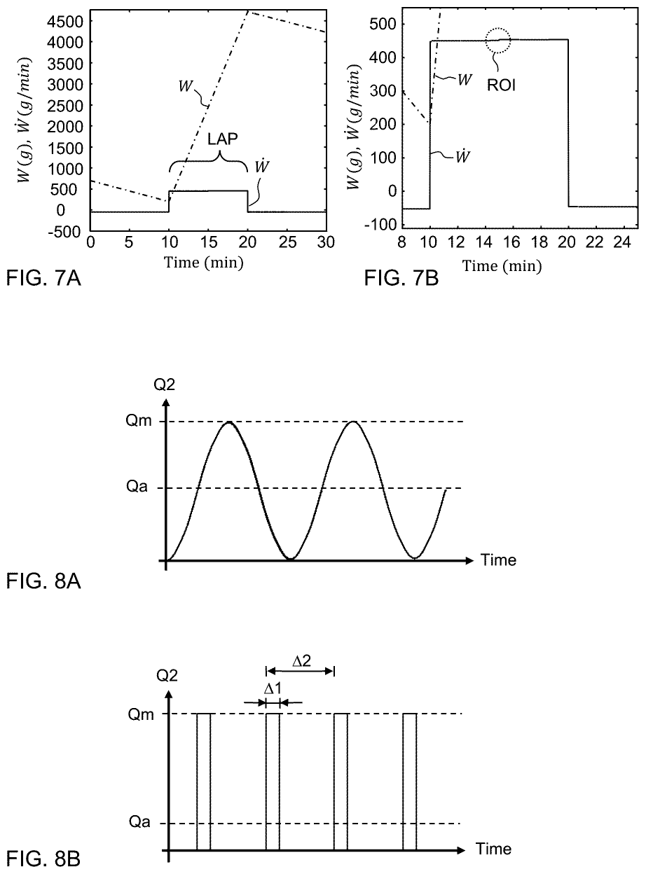

FIG. 7A is a graph of measured weight W of the container 12 as a function of time before, during and after an LAP. In the following discussion, it is assumed that the fluid F1 is fresh dialysis fluid and, thus, that Q2 represents the flow rate of fluid out of the container 12. The weight W is given by the signal S1. In the illustrated example, Q2 is 50 ml/min, Q1 is 500 ml/min, and the volume of fluid F1 that is filled into the container 12 during the LAP is 5000 ml. Thereby, the duration of the LAP is 10 minutes. At 15 min in FIG. 7A, there is an incident that reduces the stroke volume of pump P2 by 5%. The incident is virtually undetectable in the weight W of the container 12 but is seen in its weight change W, as highlighted by a dashed circle ROI in the enlarged view in FIG. 7B. The weight change \dot{W} represents the momentary change in the weight W. It is realized that the detection of the step-change in step 602 may be facilitated by evaluation of weight change \dot{W} rather than weight W. In some embodiments, the scale 12 is configured to provide the signal S1 to directly represent \dot{W}. In other embodiments, the scale 12 is configured to provide the signal S1 to represent W, and step 602 comprises converting the signal S1 into W, for example by calculating a first derivative of the signal S1, for example by use of a conventional differentiation function, optionally in combination with smoothing.

The step-change may be quite small but nevertheless have a significant impact on the calculated UF parameter. In the example of FIGS. 7A-7B, the step-change in Q2 is 2.5 ml/min (5% of 50 ml/min) in relation to a net weight change of 450 ml/min. There is thus a general desire to improve detectability of step-changes in the signal S1, and possibly also to enable a reliable quantification of the step-change.

It has been found that the step-change may be magnified by imposing a predefined and repetitive temporal variation on the pumping speed of the pump P2, via the control signal C2 (FIG. 1) to thereby cause the flow rate Q2 to exhibit the repetitive temporal variation. Such a temporal variation may be imposed without changing the average flow rate of P2, and thereby without changing the UF parameter. As used herein, “temporal variation” refers to a variation that has an extent in time.

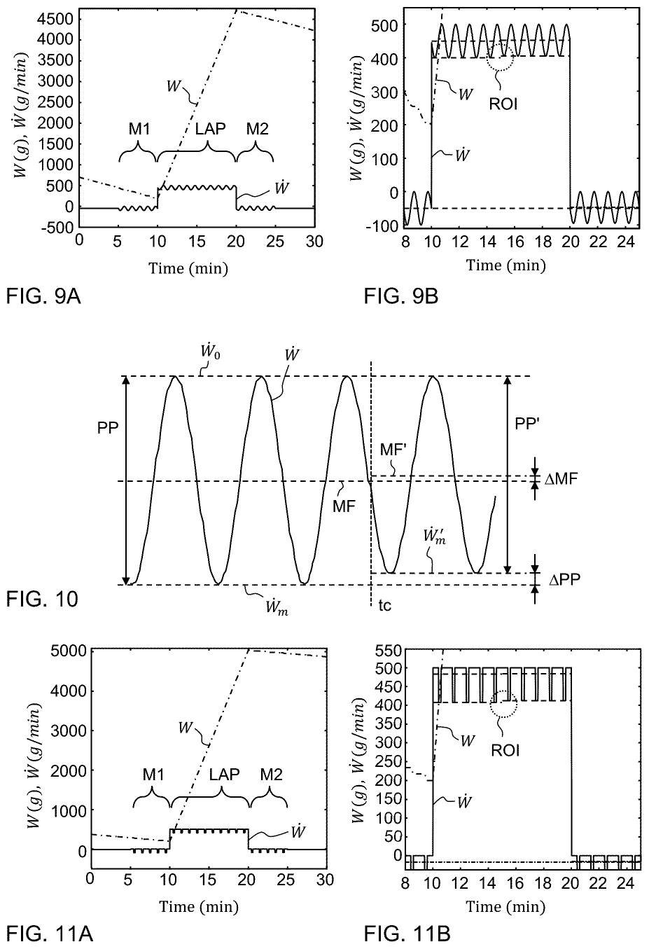

The concept of imposing the temporal variation will be described with reference FIG. 8A in combination with FIGS. 9A-9B. FIG. 8A shows an example of fluid flow rate Q2 generated by pump P2 (FIG. 1) as a function of time. As seen, the pump P2 is operated to generate a sinusoidal variation of Q2. The flow rate may be represented as Q2=Qa·(1+R·sin(2πft)), with Qa being the average flow rate, f being the frequency of the sinusoidal variation, and R being a coupling factor that controls the peak-to-peak value of the sinusoidal variation (0<R≤1). With R=1, the peak-to-peak value is maximized and Q2 varies between 0 and a maximum value Qm=2·Qa, as shown in FIG. 8A. The frequency f of the sinusoidal variation is set in view of the capabilities of the pump P2 to generate an oscillating flow rate.

FIGS. 9A-9B correspond to FIGS. 7A-7B and show measured weight W and weight change \dot{W} for the container 12 as a function of time when the flow rate Q2 has the sinusoidal variation of FIG. 8A. The average flow rate of Q2 is the same as in FIGS. 7A-7B. Like in FIGS. 7A-7B, the dashed circle ROI indicates a step-change corresponding to an incident that causes the stroke volume of pump P2 to be reduced by 5%. A further enlarged view of the ROI is shown in FIG. 10. The incidence occurs at time tc. Before the incidence, the weight change \dot{W} oscillates between a maximum value of \dot{W}₀ and a minimum value \dot{W}ₘ. Since \dot{W} is given by the difference between the inflow (Q1) of dialysis fluid into the container 12 and the outflow (Q2) of dialysis fluid from the container 12 (FIG. 1), \dot{W}₀ corresponds to Q1 (Q2=0) and \dot{W}ₘ corresponds to Q1−Qm (Q2=Qm). Before tc, the weight change \dot{W} oscillates around a mean value MF, which corresponds to Q1−Qa. At tc, MF is changed to MF′. The difference between MF and MF′, denoted ΔMF in FIG. 10, corresponds to a change in Qa and is equal in magnitude to the step-change in FIGS. 7A-7B. However, after tc, the weight change W oscillates between \dot{W}₀, which corresponds to Q1 and is unchanged, and a minimum value \dot{W}′ₘ, which differs from \dot{W}ₘ by 2·ΔMF. Thus, by imposing the sinusoidal variation, it is possible to magnify the step-change by a factor of 2. Generally, for a coupling factor R, the magnification factor is 1+R. In the specific example of FIGS. 9A-9B, the step-change is increased to 5 ml/min, compared to 2.5 ml/min in FIGS. 7A-7B.

In some embodiments, step 602 may detect and quantify, if necessary, the step-change by monitoring the peak-to-peak value of the oscillation in weight change W during the LAP. As seen in FIG. 10, the peak-to-peak value will exhibit a step-change ΔPP from PP to PP′ at time tc. The peak-to-peak value may be determined at high accuracy by evaluating \dot{W} for a time period comprising a plurality of oscillations.

Reverting to FIG. 9B, it is seen that the sinusoidal variation is imposed on the flow rate Q2 also during the measurement periods M1 and M2. This is done to maximize the likelihood that the operating conditions of the pump P2 is the same in M1 and M2 as in the LAP, and thereby that the first and second values determined by steps 302-303 (FIG. 3) are applicable to the LAP. However, in a variant, the sinusoidal variation is imposed only in the LAP and not in M1 and M2.

As understood from the example in FIG. 8A, the magnification of the step-change scales with the amplitude of the temporal variation. Given that the average flow rate needs to be maintained compared to a non-varied fluid flow, the maximum magnification is limited to a factor of 2 for a sinusoidal variation. FIG. 8B shows an alternative temporal variation that may be imposed on the flow rate Q2, possibly to further magnify the step change. The temporal variation defines a momentary increase in Q2 to a maximum value Qm for a time period Al. The temporal variation is repeated at a period of Δ2 to generate a time sequence of separate pulses (“pulse train”). It should be understood that FIG. 8B is an idealized illustration and that the flow rate Q2 in practice may be a sequence of time-separated pulses of any shape. In FIG. 8B, the pulses are generated from a baseline flow rate that is zero, which corresponds to an intermittent activation of the pump P2 for a duration of Al. However, the baseline flow rate may differ from zero, although this will reduce the detectability of the step-change based on the amplitude of the temporal variation as represented in Q2 (cf. PP′ in FIG. 10).

An example of using a temporal variation in the form of a pulse train is shown in FIGS. 11A-11B. Like in FIGS. 9A-9B, the temporal variation is imposed not only in the LAP, but also in M1 and M2. The dashed circle ROI indicates a step-change corresponding to an incident that causes the stroke volume of pump P2 to be reduced by 5%. In the example of FIGS. 11A-11B, the amplitude of the pulses is 92.4 ml/min (Qm in FIG. 8B), the number of pulses is 1 pulse per minute (Δ2=1 minute), and the average flow rate is 17 ml/min (Qa in FIG. 8B). The magnification factor is 5.4 (92.4/17). Thus, the magnitude of the step-change is increased significantly compared to FIGS. 7A-7B.

It may be noted that the provision of a time-varying flow of dialysis fluid through the dialyzer 14 (FIG. 1), be it sinusoidal or in the form of a pulse train, is unlikely to have a significant impact on the blood treatment in the dialyzer 14 since the exchange of solutes across the semipermeable membrane 14′ will be sustained as long as there is a concentration gradient between first and second chambers of the dialyzer 14. The concentration gradient is unlikely to be affected by the pulsating flow, especially when the average flow rate of fresh dialysis fluid into the dialyzer is relatively low, for example below 50-100 ml/min. Such flow rates of dialysis fluid are typically used in acute treatment, for example CRRT (Continuous Renal Replacement Therapy).

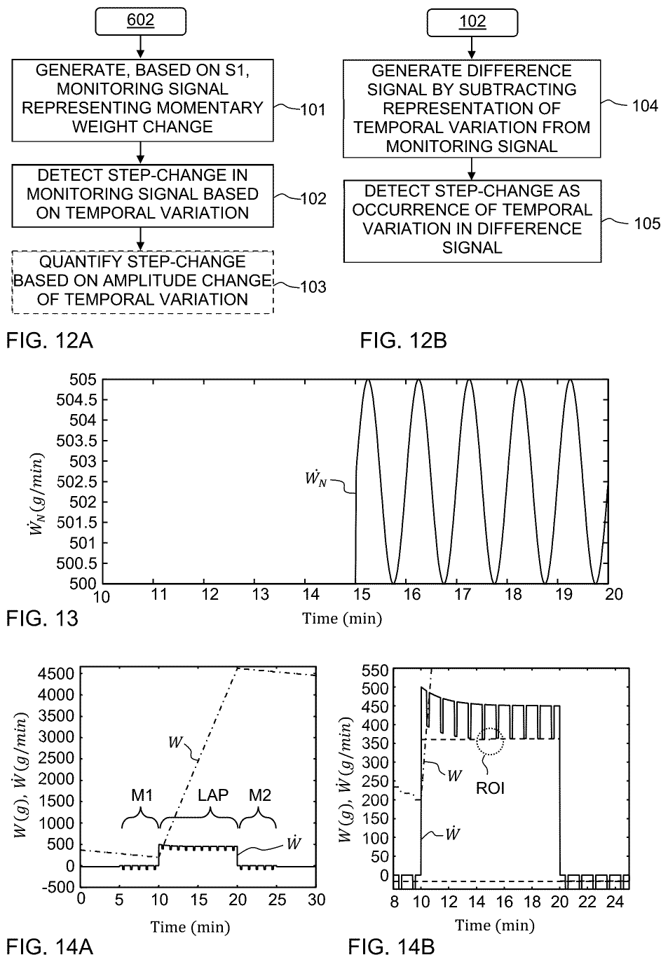

FIG. 12A is a flow chart of an example procedure that may be part of step 602 in FIG. 6, to detect a step-change in the signal S1. In step 101, a monitoring signal is generated, based on S1, to represent the momentary change of the weight of the container 12. Depending on the configuration of the scale 12, the monitoring signal may be directly given by S1 or be generated to represent a derivative of S1. As explained hereinabove, the step-change is more detectable in such a monitoring signal. Step 102 presumes that the pump P2, when operated at the known setting (cf. steps 302-304 in FIG. 3), is configured to repeatedly generate a temporal variation in fluid flow rate in the supply line 13. As understood from the foregoing, the temporal variation may comprise a minimum flow rate and a maximum flow rate. In some embodiments, the difference between the minimum flow rate and the maximum flow rate is at least twice the average flow rate that is generated by the pump P2 when operated at the known setting. As explained, this will magnify the step-change correspondingly in the signal S1. A magnification factor of 2 may be achieved by use of a sinusoidal variation. An even larger magnification factor may be achieved by implementing the temporal variation to result in a sequence of time-separated pulses of increased flow rate. In some embodiments, such a sequence is achieved by intermittent activation of the pump P2. In step 102, the monitoring signal is processed for detection of the step-change based on the temporal variation. As exemplified hereinabove, the step-change may be detected as a momentary change in the amplitude of the temporal variation as embedded in the signal S1, for example by evaluating the amplitude of the corresponding temporal variation in the monitoring signal. In step 103, which is optional, the step-change is also quantified based on the change in amplitude detected in step 102. With reference to FIG. 10, it is realized that the magnitude of the amplitude change, ΔPP, may be converted into a corresponding change of the average flow rate, ΔMF, if the temporal variation is known. The change of average flow rate may then be used by step 603 (FIG. 6).

FIG. 12B is a flow chart of an example procedure that may be part of step 102 in FIG. 12A, to further enhance the detectability of the step-change. In step 104, a difference signal is generated by subtracting a representation of the temporal variation from the monitoring signal. In step 105, the difference signal is processed for detection of the temporal variation, and the step-change is detected by occurrence of the temporal variation in the difference signal. The procedure in FIG. 12B is based on the insight that if the temporal variation is substantially cancelled in the difference signal, the temporal variation will emerge in the difference signal when a step-change occurs. The temporal variation, being known, is simple to detect. FIG. 13 is a graph of a difference signal \dot{W}_{N}, which is generated by subtracting the sinusoidal variation in FIG. 8A from the weight change \dot{W} in FIG. 9B. The difference signal \dot{W}_{N} in FIG. 13 is shown during the LAP. As seen, the temporal variation emerges in the difference signal \dot{W}_{N} when the step-change occurs at 15 minutes into the difference signal. The representation of the temporal variation, as used in step 104, may be any estimation of the temporal variation in the monitoring signal. For example, the representation may be given by a theoretical model of the monitoring signal, be generated based on the monitoring signal in a calibration session, or be generated based on the monitoring signal during the initial part of the LAP. The representation need not be an exact match of the temporal variation in the monitoring but should be sufficiently similar to cause a distinct change in the difference signal when the step-change occurs. The subtraction in step 104 comprises a phase matching between the monitoring signal and the representation of the temporal variation. The phase of the temporal variation in the monitoring signal may be known by simulation or calibration measurement. The detection of the temporal variation in step 105 may be performed by any reliable and well-known technique for detecting a periodic signal, for example frequency analysis, autocorrelation, pattern matching, etc.

All examples described with reference to the graphs in FIGS. 7A-7B, 9A-9B, 11A-11 B and 13 presume that the flow rate Q1 of the fluid F1 into the container 12 (FIG. 1) is fixed during the LAP. This is not necessary. The arrangement A1 may be deliberately or inherently configured to generate a time-varying flow of fluid F1. For example, the pump Q1 in A1 may be incapable of maintaining a fixed flow rate as the level of fluid F1 in the container 12 is increased. An example is shown in FIGS. 14A-14B, which correspond to FIGS. 11A-11B and illustrate measured weight W and weight change W when the flow rate generated by A1 exhibits a non-linear decline over the LAP. As seen in FIG. 14B, the flow rate Q1 declines from about 500 ml/min to about 450 ml/min over the LAP. The skilled person readily understands that there is a detectable change in amplitude of the temporal variation when the step-change occurs (cf. ROI in FIG. 14B) and that, optionally, a representation of the temporal variation may be subtracted from the measured weight change to further enhance detection.

Although the method 600 in FIGS. 6, 12A and 12B has been described with reference a refilling operation of the container 12 performed by Al, it is equally applicable to a draining operation of the container 16 performed by Δ2.

Further, although the method 600 of detecting a step-change has been described in combination with the method 300, the method 600 need not be combined with the method 300. Even if the time profile of the pumping parameter is not estimated in accordance with the method 300, the method 600 may be performed to detect a step-change during the LAP. If a step-change is detected, step 603 may initiate a dedicated action. Although the time profile has not been estimated by use of the method 300, step 603A may be performed to account for the step-change in a nominal time profile of the pumping parameter. Alternatively or additionally, step 603B may be performed to modify the LAP, for example by stopping the LAP, and/or step 603C may be performed to modify the pumping speed of pump P2/P3 to at least partly compensate for the change in flow rate Q2/Q3 caused by the step-change.

Further, instead of detecting the step-change in the weight signal from a scale, steps 601-602 in FIG. 6 may detect the step-change based on a signal indicative of the performance of the pump P2 or P3. For example, the step-change may be inferred from the power consumption, drive current, torque, speed, etc., of the pump P2 or P3.

While the subject of the present disclosure has been described in connection with what is presently considered to be the most practical and preferred embodiments, it is to be understood that the subject of the present disclosure is not to be limited to the disclosed embodiments, but on the contrary, is intended to cover various modifications and equivalent arrangements included within the spirit and the scope of the appended claims.

Further, while operations are depicted in the drawings in a particular order, this should not be understood as requiring that such operations be performed in the particular order shown or in sequential order, or that all illustrated operations be performed, to achieve desirable results. In certain circumstances, parallel processing may be advantageous.

1: A device for monitoring ultrafiltration in a system for extracorporeal treatment of blood, the device comprising:
an input interface, which is configured to receive a measurement signal representative of a momentary weight of a container in the system, the container being connected on a first fluid path to a first dialyzer port for dialysis fluid on a dialyzer for extracorporeal treatment of blood, a pumping device being arranged in the first fluid path , the container being further connected on a second fluid path to an adjustment arrangement for level adjustment of a fluid in the container, and
processor circuitry, which is connected to the input interface and configured to perform a monitoring procedure in relation to a level adjustment period, in which the adjustment arrangement is selectively activated to perform the level adjustment and the pumping device is operated at a known setting,
wherein the processor circuitry, in the monitoring procedure, is configured to:
determine, during a first measurement period prior to the level adjustment period and based on the measurement signal, a first value of a pumping parameter of the pumping device while the adjustment arrangement is deactivated and the pumping device is operated at the known setting,
determine, during a second measurement period subsequent to the level adjustment period and based on the measurement signal, a second value of the pumping parameter while the adjustment arrangement is deactivated and the pumping device is operated at the known setting,
estimate, based on the first and second values, a time profile of the pumping parameter in the level adjustment period, and
determine an ultrafiltration parameter for the level adjustment period based on the time profile.

2: The device of claim 1, wherein the processor circuitry is configured to estimate the time profile by performing a linear interpolation of the first and second values.

3: The device of claim 1, wherein the pumping parameter is one of: a flow rate generated by the pumping device or a stroke volume of the pumping device.

4: The device of claim 1, wherein the pumping parameter is a stroke volume of the pumping device, and wherein the processor circuitry is further configured to receive, via the input interface, a further measurement signal representative of a speed of the pumping device, and determine the first and second values of the pumping parameter based on the measurement signal and the further measurement signal during the first and second measurement period, respectively.

5: The device of claim 4, wherein the processor circuitry is further configured to: determine a fluid flow parameter for the level adjustment period, based on the time profile and the further measurement signal during the level adjustment period, and determine the ultrafiltration parameter based on the fluid flow parameter.

6: The device of claim 5, wherein the fluid flow parameter represents a total amount of the fluid pumped by the pumping device during the level adjustment period.

7: The device of claim 6, wherein the processor circuitry is configured to:
determine, based on the further measurement signal, a count of pumping strokes performed by the pumping device during the level adjustment period, calculate an average stroke volume for the level adjustment period based on the time profile, and determine the fluid flow parameter as a function of the average stroke volume and the count of the pumping strokes.

8: The device of claim 4, wherein the further measurement signal comprises a predefined number of pulses for each pumping stroke of the pumping device.

9: The device of claim 1, wherein the ultrafiltration parameter comprises an amount of ultrafiltrate extracted from the blood in the dialyzer.

10: The device of claim 1, wherein the processor circuitry is configured to:
receive, via the input interface, an input signal which is representative of fluid pressure in the first fluid path between the container and the pumping device or which is representative of a control signal for the pumping device, and
estimate the time profile based on the first and second values and the input signal during the level adjustment period.

11: The device of claim 10, wherein the processor circuitry is further configured to:
obtain calibration data representing a relation between the fluid pressure and the pumping parameter; and
estimate the time profile by converting, by use of the calibration data, the input signal received during the level adjustment period into values of the pumping parameter.

12: The device of claim 1, wherein the processor circuitry is further configured to:
monitor the measurement signal for detection of a step-change during the level adjustment period, and
to initiate, upon detection of the step-change, a dedicated action.

13: The device of claim 12, wherein the dedicated action comprises one or more of: a modification of the level adjustment period, accounting for the step-change when estimating the time profile of the pumping parameter, or a modification of a pumping speed of the pumping device.

14: The device of claim 13, wherein the modification of the level adjustment period results in a stop of the level adjustment.

15: The device of claim 13, wherein the processor circuitry is configured to, upon the detection of the step-change, determine a magnitude of the step-change, wherein the dedicated action uses the magnitude.

16: The device of claim 15, wherein the modification of the pumping speed is based on the magnitude to at least partly compensate for a change in flow rate in the first fluid path resulting from the step-change.

17: The device of claim 15, wherein the processor circuitry is configured to estimate the time profile of the pumping parameter based on the first and second values, the magnitude, and a timing of the step-change during the level adjustment period.

18: The device of claim 12, wherein the processor circuitry is configured to generate, based on the measurement signal, a monitoring signal that represents momentary change of the weight of the container, and detect the step-change in the monitoring signal.

19: The device of claim 12, wherein the pumping device, when operated at the known setting, is configured to repeatedly generate a temporal variation in fluid flow rate in the first fluid path, and wherein the processor circuitry is configured to detect the step-change as a momentary change in an amplitude of the temporal variation as embedded in the measurement signal.

20: The device of claim 19, wherein the processor circuitry is configured to generate, based on the measurement signal, a monitoring signal that represents a momentary change of the weight of the container, and detect the step-change based on the monitoring signal.

21: The device of claim 20, wherein the processor circuitry is configured to generate the monitoring signal to represent a derivative of the measurement signal.

22: The device of claim 20, wherein the processor circuitry is configured to generate a difference signal by subtracting a representation of the temporal variation from the monitoring signal and detect the step-change in the difference signal.

23: The device of claim 22, wherein the processor circuitry is configured to process the difference signal for detection of the temporal variation, wherein the step-change is detected by presence of the temporal variation in the difference signal.

24: The device of claim 19, wherein the temporal variation comprises a minimum flow rate and a maximum flow rate.

25: The device of claim 24, wherein a difference between the minimum flow rate and the maximum flow rate is at least twice an average flow rate generated by the pumping device when operated at the known setting.

26: The device of claim 19, wherein the temporal variation is a sinusoidal variation.

27: The device of claim 19, wherein the temporal variation results in a sequence of time-separated pulses of increased flow rate.

28: The device of claim 27, wherein the temporal variation corresponds to an intermittent activation of the pumping device.

29: The device of claim 1, wherein the fluid in the container is fresh dialysis fluid for use in the extracorporeal treatment of blood in the dialyzer, or water that is mixed with one or more concentrates in the first fluid path to form the fresh dialysis fluid, and wherein the first dialyzer port is an inlet port for the fresh dialysis fluid.

30: The device of claim 1, wherein the fluid in the container is spent dialysis fluid resulting from the extracorporeal treatment of blood in the dialyzer, and the second dialyzer port is an outlet port for the spent dialysis fluid.

31: The device of claim 1, wherein the processor circuitry is further configured to generate control signals for the pumping device, the adjustment arrangement, and a further pumping device.

32: The device of claim 1, wherein the ultrafiltration parameter is further determined based on fluid flow data representing fluid flow generated in the level adjustment period by one or more other pumping devices in direct or indirect fluid communication with blood in an extracorporeal blood circuit connected to the dialyzer.

33: A device for controlling flow rate in a system for extracorporeal treatment of blood, the device comprising:
an input interface, which is configured to receive a measurement signal representative of a momentary weight of a container in the system, the container being connected on a first fluid path to a first dialyzer port for dialysis fluid on a dialyzer for extracorporeal treatment of blood, a pumping device being arranged in the first fluid path, the container being further connected on a second fluid path to an adjustment arrangement for level adjustment of a fluid in the container, and
processor circuitry, which is connected to the input interface and configured to perform a control procedure in relation to a level adjustment period, in which the adjustment arrangement is selectively activated to perform the level adjustment and the pumping device is operated at a known setting,
wherein the processor circuitry, in the control procedure, is configured to, during the level adjustment period, process the measurement signal for detection of a step-change, and initiate a dedicated action upon detection of the step-change.

34: A system for extracorporeal blood treatment, the system comprising:
a dialyzer comprising first and second dialyzer ports for dialysis fluid,
a container connected on a first fluid path to the first dialyzer port on the dialyzer,
a pumping device in the first fluid path,
an adjustment arrangement for level adjustment of a fluid in the container, the adjustment arrangement being connected to the container on a second fluid path,
a weighing device arranged to generate a measurement signal representative of a momentary weight of the container, and
a device comprising:
an input interface, which is configured to receive a measurement signal representative of a momentary weight of the container in the system, and
processor circuitry, which is connected to the input interface and configured to perform a control procedure in relation to a level adjustment period.

35: A computer-implemented method of monitoring ultrafiltration in a system for extracorporeal treatment of blood, the method comprising:
receiving a measurement signal representative of a momentary weight of a container in the system, the container being connected on a first fluid path to a first dialyzer port for dialysis fluid on a dialyzer for extracorporeal treatment of blood, a pumping device being arranged in the first fluid path, the container being further connected on a second fluid path to an adjustment arrangement for level adjustment of a fluid in the container; and
performing a monitoring procedure in relation to a level adjustment period, in which the adjustment arrangement is selectively activated to perform the level adjustment and the pumping device is operated at a known setting;
wherein the monitoring procedure comprises:
determining, during a first measurement period prior to the level adjustment period and based on the measurement signal, a first value of a pumping parameter of the pumping device while the adjustment arrangement is deactivated and the pumping device is operated at the known setting;
determining, during a second measurement period subsequent to the level adjustment period and based on the measurement signal, a second value of the pumping parameter while the adjustment arrangement is deactivated and the pumping device is operated at the known setting;
estimating, based on the first and second values, a time profile of the pumping parameter in the level adjustment period; and
determining an ultrafiltration parameter for the level adjustment period based on the time profile.

36: A computer-implemented method of controlling flow rate in a system for extracorporeal treatment of blood, the method comprising:
receiving a measurement signal representative of a momentary weight of a container in the system, the container being connected on a first fluid path to a first dialyzer port for dialysis fluid on a dialyzer for extracorporeal treatment of blood, a pumping device being arranged in the first fluid path, the container being further connected on a second fluid path to an adjustment arrangement for level adjustment of a fluid in the container; and
performing a control procedure in relation to a level adjustment period, in which the adjustment arrangement is selectively activated to perform the level adjustment and the pumping device is operated at a known setting;
wherein the control procedure comprises:
processing, during the level adjustment period, the measurement signal for detection of a step-change; and
initiating a dedicated action upon detection of the step-change.

37: A computer-readable medium comprising program instructions, which when executed by a processor causes the processor to perform a method comprising:
receiving a measurement signal representative of a momentary weight of a container in a system for extracorporeal treatment of blood, the container being connected on a first fluid path to a first dialyzer port for dialysis fluid on a dialyzer for extracorporeal treatment of blood, a pumping device being arranged in the first fluid path, the container being further connected on a second fluid path to an adjustment arrangement for level adjustment of a fluid in the container; and
performing a monitoring procedure in relation to a level adjustment period, in which the adjustment arrangement is selectively activated to perform said level adjustment and the pumping device is operated at a known setting;
wherein the monitoring procedure comprises:
determining, during a first measurement period prior to the level adjustment period and based on the measurement signal, a first value of a pumping parameter of the pumping device while the adjustment arrangement is deactivated and the pumping device is operated at the known setting;
determining, during a second measurement period subsequent to the level adjustment period and based on the measurement signal, a second value of the pumping parameter while the adjustment arrangement is deactivated and the pumping device is operated at the known setting;
estimating, based on the first and second values, a time profile of the pumping parameter in the level adjustment period; and
determining an ultrafiltration parameter for the level adjustment period based on the time profile.
